(19) United States
(12) Patent Application Publication
(10) Pub. No.: US 2012/0074334 A1
(43) Pub. Date: Mar. 29, 2012

(54) Sterilization of household and other article

(76) Inventor: Gregory P. Milligan, Weston, FL (US)

(21) Appl. No.: 13/200,255

(22) Filed: Sep. 22, 2011

Related U.S. Application Data
(60) Provisional application No. 61/403,995, filed on Sep. 25, 2010.

(51) Int. Cl. A61L 2/10

(52) U.S. Cl. 250/455.11

(57) ABSTRACT

An apparatus for sterilizing includes receptacle having a base and left and right end surfaces; a cover for closing the apparatus, the cover having an outer surface, an inner surface and left and right end surfaces; a first UV light source positioned on the inner surface of the cover; a second UV light source positioned within the receptacle; and at least one article-receiving panel positioned over the second light source of the base of the receptacle.

### REFERENCE TO RELATED APPLICATION

This application claims the benefit under 35 U.S.C. 119 (e) of provisional application Ser. No. 61/403,995, filed Sep. 25, 2010, which is hereby incorporated by reference in its entirety.

### BACKGROUND OF THE INVENTION

A. Area of Invention

The present invention relates to sterilization, and more particularly, to sterilization of household and other articles by employing ultraviolet (UV) radiations.

B. Prior Art

In past few decades, there has been a widespread increase in awareness of the potentially harmful effects that microorganisms, such as fungi, bacteria and viruses can have on humans. Consequently, there have been several attempts to provide devices that sterilize food, liquids, toys, toothbrush and other similar items.

Generally, anti-bacterial products are employed to fight against transmittal of germs. For example, many hand soaps have added anti-bacterial ingredients. Sterilization devices are primarily aimed at sanitization of objects that may have bacteria upon their surface. Additionally, such sterilization devices are necessary for all age groups, such as, for example, infants, wherein minimizing the presence of bacteria is particularly important because they are more susceptible to infection. The risk of infection increases further as infants have a tendency to place toys and other objects, potentially contaminated with undesirable pathogen, within their mouths. Typically, undesirable pathogen that cause such infection include: Streptococcus Group B, Streptococcus Group A, Hemopholis Influenza, Streptococcus Pneumonia, Marsa, Pseudomonas Aeruginosa, influenza virus and the like.

Conventionally, several approaches for sanitizing surfaces of personal hygiene articles may be known, but, all known conventional methods and devices have one or more disadvantages.

One traditional approach includes soaking the articles (such as toothbrush), in a chemical disinfectant. However, chemical disinfectants may not reach all of the surfaces of the articles, such as the surfaces between tightly compacted bristles found in many toothbrushes. Furthermore, this approach may be ineffective especially if the exposure of the articles to the chemical disinfectant is brief. In addition, such chemical disinfectants may be relatively costly and may have to be frequently resupplied. Accordingly, use of chemical disinfectants may not be the entirely preferred one as it suffers from various disadvantages.

Previous approaches further include sanitization of personal hygiene articles by employing microwave energy. However, it may be problematic because of the electrical arcing that may occur with metal used in the articles. Moreover, most households may not be equipped with a microwave apparatus outside of the kitchen. Therefore, use of this approach may be inconvenient for a majority of household users.

In addition to the above, a common problem encountered by almost all the currently known approaches is that contaminants may be allowed to enter the system during and/or after the sanitization process. This may reverse the effects of the sanitization process, thereby making the sanitization process less effective.

The general use of UV sterilizers by physical therapists, aestheticians and other such professionals wishing to sterilize their tools and equipment has long been known in the art. The preferred frequency of UV light used therein is 254 nanometers. As above noted, such general purpose sterilizers are not optimal in terms of internal or external geometry for the specific purposes as described above. Prior art patents or patent literature exists for certain special-purpose UV products, such as United States Patent Application Publication U.S. 2008/0044326 A1 (2008) to Shin, entitled Sterilizer for Baby Products. This sterilization system is applicable for only short range, i.e., in the order of 1 to 2 centimeters from the UV source. Similarly, WIPO Application Publication No. WO 02/056920 A2 (2002) to Jenkins et al, entitled Method and Apparatus for Rapidly Sterilizing Small Objects is, similarly to the disclosure of Shin, adapted for short range sterilization and, as in Shin, lacks any internal reflective surfaces which could operate to enhance the effect of the applied radiation. U.S. Pat. No. 6,039,928 (2000) to Roberts, entitled Writing Implement Sterilization Apparatus is a special purpose UV sterilization device applicable in geometry and other respects only to the sterilization of writing implements.

Accordingly, there exists a need for sterilizers for sanitizing multiple articles, which are free from above-mentioned problems associated with existing techniques, and provide additional advantages in terms of effectiveness, size, cost, safety, usage convenience and the like.

### SUMMARY OF THE INVENTION

An apparatus for sterilizing household articles includes a receptacle having a base, and left and right ends thereof; a cover for closing the apparatus, the cover having an outer surface, an inner surface and left and right ends; at least a first UV light source positioned on the inner surface of the cover; at least a second UV light source positioned within the receptacle; and at least one article-receiving panel positioned over said second light source of said base surface of said receptacle.

It is an object of the present invention to provide an improved system for the sterilization of household and other articles that is not inherently limited to the geometry or size of the article to be sterilized and includes upper and lower UV light sources internal to the sterilizing apparatus while providing flexibility in the manner of positioning within the sterilizing receptacle of the articles to be sterilized.

Another object is to provide an apparatus of the above type in which the effectiveness of the UV radiation emitted by the UV light source of the invention is enhanced through the use of reflective surfaces internal to the apparatus.

It is a yet further object of the invention to provide an apparatus of the above type in which internal supportive panels or shelves thereof are UV transmissive in order to enhance the effect of UV radiation upon the articles to be sterilized.

The above and yet other objects and advantages of the present invention will become apparent from the herein set forth Brief Description of the Drawings, Detailed Description of the Invention, and Claims appended herewith.

### BRIEF DESCRIPTION OF THE DRAWINGS

FIG. 1 illustrates an apparatus for sterilizing multiple articles, according to an embodiment of the present invention.

FIG. 2 illustrates the apparatus for sterilizing multiple articles in an open position.

FIG. 3 illustrates a front view of the apparatus for sterilizing multiple articles.

FIG. 4 illustrates a cross sectional view of the apparatus for sterilizing multiple articles, according to an embodiment of the present invention.

FIG. 5 illustrates a cross sectional view of the apparatus for sterilizing multiple articles.

### DETAILED DESCRIPTION OF THE INVENTION

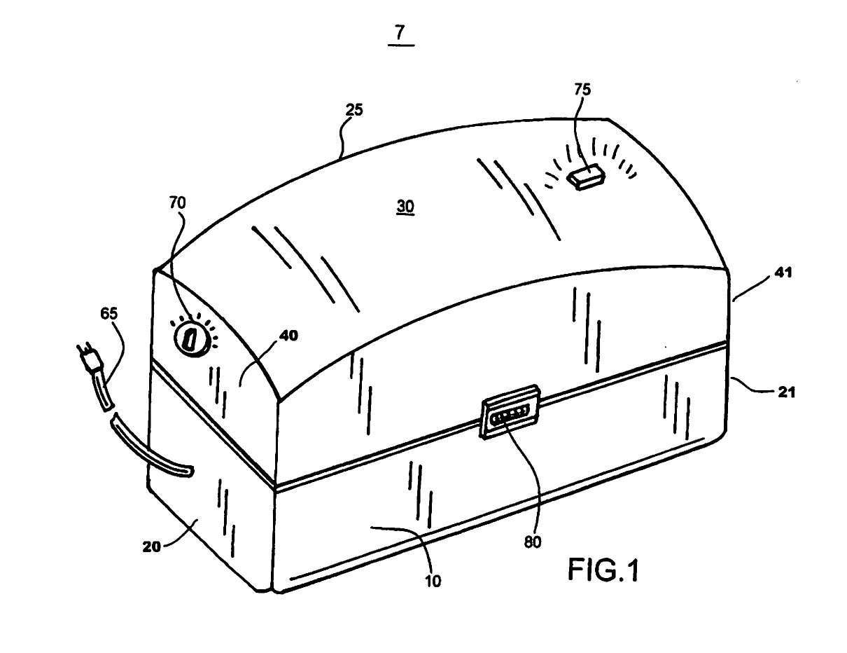

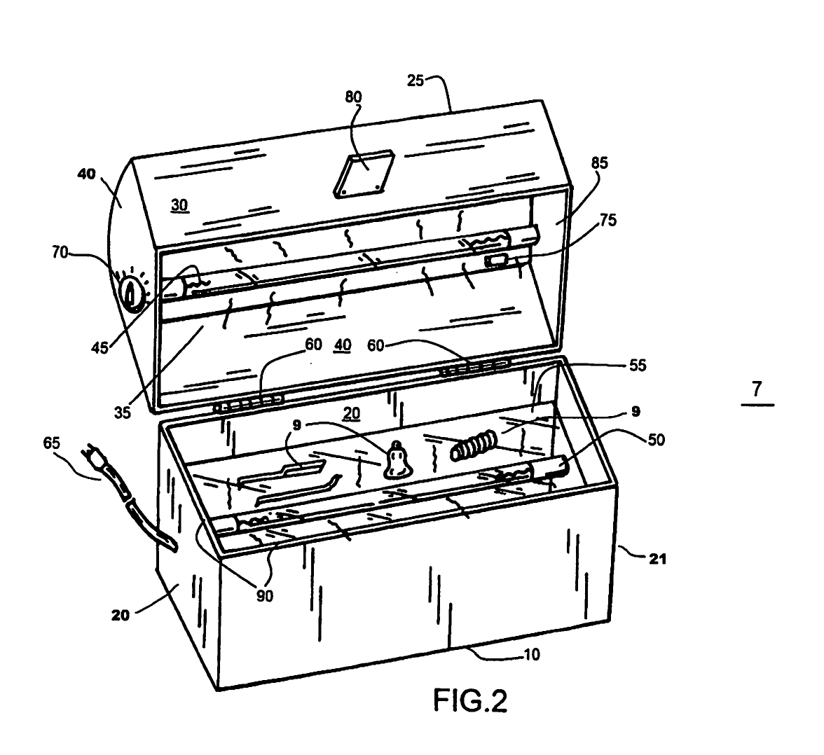

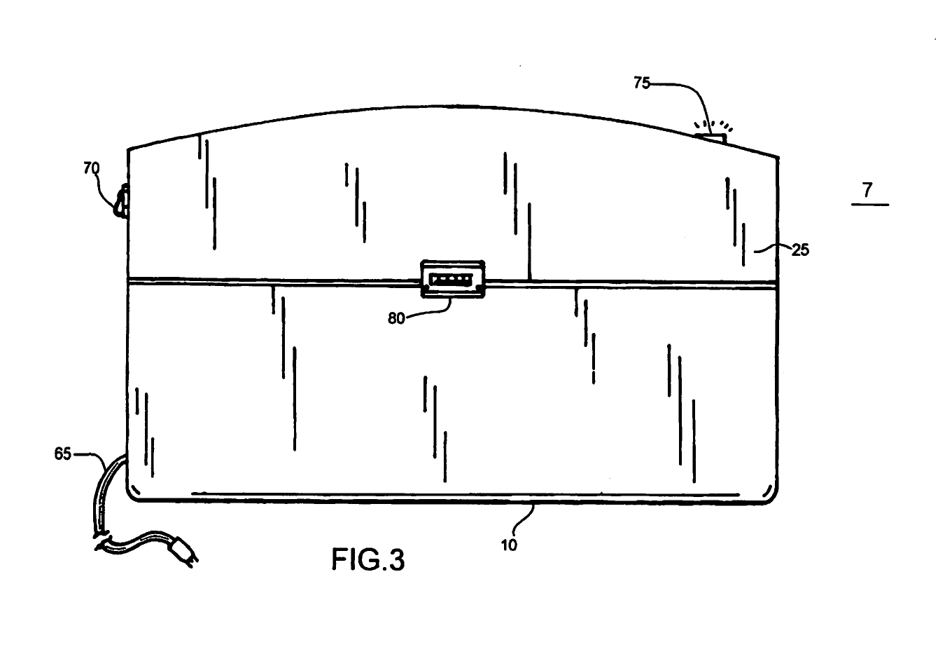

As may be seen in FIGS. 1-5, there is provided an apparatus 7 for sterilizing various articles 9, which includes a receptacle 10 having a base 15 and end regions 20 and 21, as well as a cover 25 for covering the receptacle 10. The cover member 25 includes an outer surface 30, an inner surface 35 and end regions 40 and 41. The apparatus further includes a first light source 45 positioned on the inner surface 35; which is preferably curved, of the cover 25, a second light source 50 positioned on the base 15 of the receptacle 10, and a panel member 55 positioned over the second light source 50 of the base 15 of the receptacle 10. See FIG. 2. Generally, the inner surface 35 of the covering member 25 and the base 15 of the receptacle 10 are reflective in nature. Furthermore, the cover 25 is probably curvilinear in shape and an optionally height adjustable panel member 55 is a quartz sheet, or other UV transmissive material. It will be appreciated that more than one light source 45/50 may be employed in the cover 25 and/or in the receptacle 10 depending upon the requirements of the sterilizing multiple articles 9.

According to an aspect of the present invention, the various articles 9 may include keys, cell phones, television remote controllers, video games, and the other similar household items or non-household items.

According to another aspect of the present invention, the receptacle 10 is securable to the cover 25 via a coupling member 80 or the like. Generally, the coupling member is a hinge, and the end regions 20/21 of the receptacle 10 includes a half of the hinge mounted on an upper edge of the receptacle 20, and the cover 25 includes a second half of the hinge 80 mounted on an edge of the cover opposite said first half of the hinge. Subsequently, the first half of the hinge mates with the second half, thereby forming the coupling member 60. However, other similar coupling members may also be employed. That is, the apparatus for sterilizing multiple articles 9 further may include a lock for locking the cover member 25 to the receptacle 10. Generally, such a lock includes a clasp to lock the cover member 25 and the receptacle 10 in a closed position. Other similar locking mechanisms may also be employed.

According to a further aspect of the invention, the first light source 45 and the second light source 50 are connected to a power source 65 capable of powering a UV light output, e.g., 1800 watts. Generally, UV light kills micro-organisms by damaging their DNA or RNA. More specifically, UV radiation disrupts the chemical bonds that hold the sub-units or segments of DNA or RNA together in the micro-organism. Consequently, the micro-organisms cannot repair such damage and due to lack of proper functioning of their reproductive mechanisms, they die or are rendered inert.

According to another aspect of the invention, the apparatus for sterilizing multiple articles 9 further includes a power circuit timer 70 (see FIG. 1) for providing a timed application of power from the power source 65 to the first light source 45 and the second light source 50, and an indicating element 75 mounted on the outer surface 30 of the covering member 25. The indicating member 75 provides an indication whether the first light source 45 and/or the second light source 50 are in operation or not. Generally, the indicating member 75 is translucent, i.e. the indicating element 75 is partially shaded with a view to prevent harmful radiation from leaking-out of the apparatus.

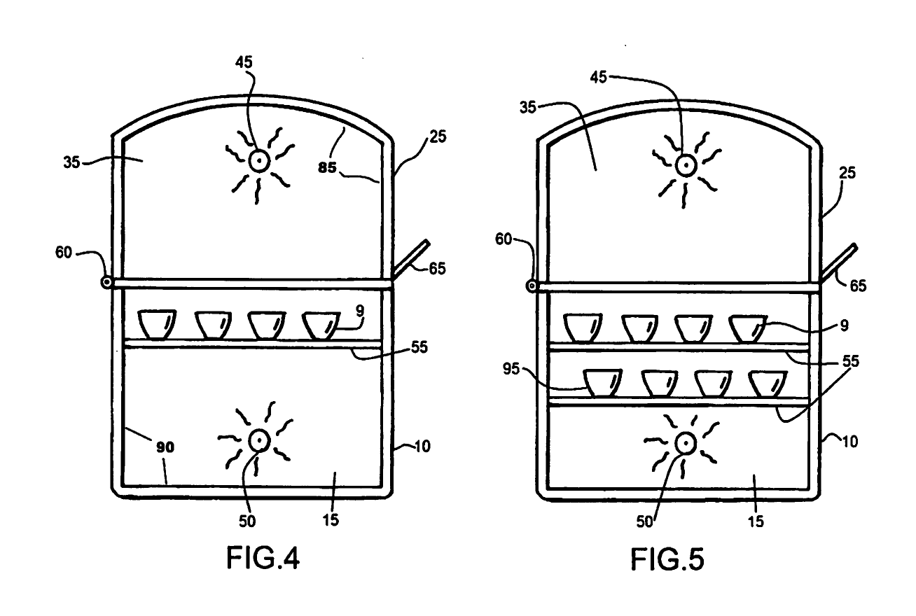

In a further aspect of the invention, the apparatus for sterilizing multiple articles 9 further includes a first padding surface 85 and/or a second padding surface 90 (see FIG. 4) for preventing leakage of radiation, in which the first padding surface 85 and the second padding surface 90 line the interiors of the covering member 25 and the receptacle 10 respectively. Generally, both padding surfaces are rubber or rubber-like in character.

In an alternate embodiment, as illustrated in FIG. 5, the apparatus for sterilizing multiple articles 9 includes more than one panel members 55 positioned over the second light source 50 of the base surface 15 of the receptacle 10. Those of ordinary skill will appreciate that multiple panel members 55 may further be employed, thereby providing provisions for sterilizing large number of articles simultaneously.

Those of ordinary skill will appreciate that the above-mentioned apparatus for sterilizing multiple articles also may be designed in a manner such that it opens from the front, instead of the top, and various such alternate embodiments may be designed as per the requirements of a user.

Accordingly, while there has been shown and described the preferred embodiment of the invention, it is to be appreciated that the invention may be embodied otherwise than is herein specifically shown and described and, within said embodiment, certain changes may be made in the form and arrangement of the parts without departing from the underlying ideas or principles of this invention within the scope of the Claims appended herewith.

I claim:

1. An apparatus for sterilizing a plurality of articles, the apparatus comprising:
(a) a receptacle having a base, and left and right ends thereof;
(b) a cover for closing said apparatus, said cover having an outer surface, an inner surface and left and right ends thereof;
(c) at least a first UV light source positioned on said inner surface of said cover member;
(d) at least a second UV light source positioned within said receptacle; and
(e) at least one UV transmissive article-receiving panel positioned over said second light
source of said base of said receptacle.

2. The apparatus as recited in claim 1, including securing means for self-locking of said receptacle to said cover.

3. The apparatus as recited in claim 1, in which said first light source and said second light source define electrical communications to a power supply capable of powering said light sources.

4. The apparatus as recited in claim 3, including said apparatus for sterilizing a plurality of articles, further comprises:
(f) a power circuit timer for providing a timed application of power from said power source to said first light source and to said second light source;
(g) an indicating member mounted on said outer surface of said cover, said indicating member providing an indication either or both first light source or said second light source are in operation; and
(h) a first padding element and a second padding element for preventing leakage of radiation from the apparatus, said first padding element and said second padding element lining interiors of said cover and said receptacle respectively.

5. The apparatus as recited in claim 4, in which said inner surface of the covering member defines a curved surface.

6. The apparatus as recited in claim 4, in which said base of the apparatus includes a curved surface.

7. The apparatus as recited in claim 1, further including at least one repositionable panel within said receptacle, said panel substantially parallel with said base.

8. The apparatus as recited in claim 1, in which said base of said receptacle and said inner surface of said cover comprise a UV reflective material.

9. The apparatus as recited in claim 5, in which said base of the apparatus includes a curved surface.

10. The apparatus as recited in claim 4, further including a translucent non-UV transmissive shield for said covering indicating member.

11. The apparatus as recited in claim 4, in which said first and second padding element within said cover and receptacle respectively each define a rubber-like material.

12. The apparatus as recited in claim 5, said cover situated at a front of the apparatus to define a front-opening structure.

13. The apparatus as recited in claim 7, in which said at least one panel member positioned over said second light source is UV transmissive.

14. The apparatus as recited in claim 5, in which said base of said receptacle and said inner surface of said cover each comprise a UV reflective material.
